(19) United States
(12) Patent Application Publication
(10) Pub. No.: US 2021/0322758 A1
(43) Pub. Date: Oct. 21, 2021

(54) USE OF GRAPHITE TO SPREAD HEAT INSIDE DEVICE

(71) Applicant: Medtronic, Inc., Minneapolis, MN (US)

(72) Inventors: Thomas J. FICK, Minneapolis, MN (US); Harold M. DYALSINGH, Brooklyn Park, MN (US); Brian D. KUHNLEY, Delano, MN (US); Jeffrey A. SWANSON, Cambridge, MN (US); Ramesh RAGHUPATHY, New Brighton, MN (US); Amir ZAMIRI, Columbia Heights, MN (US); Thomas W. RADTKE, Ramsey, MN (US); Lisa A. MEYER, Faribault, MN (US)

(21) Appl. No.: 17/208,114

(22) Filed: Mar. 22, 2021

Related U.S. Application Data
(60) Provisional application No. 63/012,987, filed on Apr. 21, 2020.

(51) Int. Cl. A61M 60/90; A61M 60/122; A61M 60/50; H01L 23/373

(52) U.S. Cl. CPC A61M 60/90; A61M 60/122; A61M 60/50; A61M 60/876; A61M 2205/02; A61M 2205/04; H01L 23/3732

(57) ABSTRACT

An implantable controller for an implantable medical device includes a metallic housing defining an enclosure. Processing circuitry is disposed within the enclosure and configured to control operation of the implantable medical device. A first aluminum encasement is disposed within the enclosure. A first piece of graphite is disposed within the aluminum encasement. A pressure sensitive adhesive is disposed between an internal surface of the metallic housing and the aluminum encasement.

### CROSS-REFERENCE TO RELATED APPLICATION

This application claims the benefit of U.S. application Ser. No. 63/012,987, filed Apr. 21, 2020.

### FIELD

The present technology is generally related to controllers for implantable medical devices.

### BACKGROUND

A mechanical circulatory support device (MCSD) such as a left ventricular assist device (LVAD) is an implantable device that is used to assist the functioning of a failing heart. LVADs include a pump that connects the left ventricle to the aorta which pulls blood from the left ventricle and pumps it into the aorta. With the advent of fully implantable systems having implantable blood pumps, such as LVADs, more electronic equipment is implanted within the body and within or proximate various types of tissue. In particular, transcutaneous energy transfer (TET) systems are used to supply power MCSDs implanted within a human body. An electromagnetic field generated by a transmitting coil outside the body can transmit power across a cutaneous (skin) barrier to a receiving coil implanted within the body. The receiving coil can then transfer the received power to the implanted heart pump or other internal device and to one or more batteries implanted within the body.

One of the challenges with MCSD systems is the fact that the controller used to power the implanted pump is also implanted within the body. Such controllers interact with various tissues within the body and because they provide power to the implanted pump, they also generate heat. If too much heat is released from the controller, or a particular part of the controller, there is potential for tissue surrounding the controller to be damaged in addition to damage to the controller itself.

### SUMMARY

The techniques of this disclosure generally relate to controllers and implantable controllers for implantable medical devices.

In one aspect, an implantable medical device includes a metallic housing defining an enclosure. Processing circuitry is disposed within the enclosure and configured to control operation of the implantable medical device. A first thermally conductive encasement is disposed within the enclosure. A first piece of graphite is disposed within the aluminum encasement. An adhesive is disposed between an internal surface of the metallic housing and the aluminum encasement.

In another aspect of this embodiment, the first piece of graphite is of annealed pyrolytic graphite.

In another aspect of this embodiment, the device further includes a battery disposed within the housing, wherein the battery is configured to provide power to the processor and to provide power to the implantable medical device.

In another aspect of this embodiment, the enclosure further includes a second thermally conductive encasement and a second piece of graphite enclosed within the second thermally conductive encasement, wherein the battery and the processing circuitry are disposed between the first thermally conductive encasement and the second thermally conductive encasement.

In another aspect of this embodiment, the device further includes a first conductive gap pad disposed between the processing circuitry and the first aluminum encasement.

In another aspect of this embodiment, the metallic housing is composed of a biocompatible metal.

In another aspect of this embodiment, the first thermally conductive encasement includes aluminum foil, and wherein the first piece graphite is laminated in the aluminum foil.

In another aspect of this embodiment, the medical device is controller for an implantable blood pump.

In another aspect of this embodiment, the first piece of graphite defines a first plane and the pressure sensitive adhesive defines a second plane substantially parallel to the first plane.

In another aspect of this embodiment, the housing is sized to be implanted within a portion of a patient's torso.

In one aspect, an implantable controller for an implantable medical device includes a metallic housing defining an enclosure, the housing is sized to be implanted within a portion of a patient's torso. The enclosure contains processing circuitry configured to control the operation of the implantable medical device. A first piece of graphite is disposed within the housing, the first piece of graphite being enclosed within a first aluminum encasement, the first aluminum encasement defining a plurality flared aluminum edges extending from the encasement. A pressure sensitive adhesive is disposed between an internal surface of the metallic housing and the first aluminum encasement, the pressure sensitive adhesive being in direct contact with the housing and with the first aluminum encasement.

In another aspect of this embodiment, the first piece of graphite is annealed pyrolytic graphite.

In another aspect of this embodiment, the controller further includes a battery disposed within the housing, wherein the battery is configured to provide power to the processor and to provide power to the medical device.

In another aspect of this embodiment, the enclosure further includes a second aluminum encasement and a second piece of graphite enclosed within the second aluminum encasement, wherein the battery and the processing circuitry are disposed between the first aluminum encasement and the second aluminum encasement.

In another aspect of this embodiment, the controller further includes a first conductive gap pad disposed between the processing circuitry and the first aluminum encasement.

In another aspect of this embodiment, the metallic housing is composed of a biocompatible metal.

In another aspect of this embodiment, the first aluminum encasement includes aluminum foil, and wherein the first piece of graphite is laminated in the aluminum foil.

In another aspect of this embodiment, the medical device is an implantable blood pump.

In another aspect of this embodiment, the first piece of graphite defines a first plane and the pressure sensitive adhesive defines a second plane substantially parallel to the first plane.

In one aspect, an implantable controller for an implantable blood pump includes a titanium housing defining an enclosure, the housing being sized to be implanted within a portion of a patient's torso. The housing contains processing circuitry configured to control the operation of the implantable blood pump and a battery configured to provide power to the implantable blood pump. A piece of graphite is disposed within the housing, the piece of graphite being enclosed and laminated within an aluminum foil encasement, the aluminum foil encasement defining a plurality flared aluminum edges extending from the encasement and contouring a corresponding curved interior surface of the housing. A conductive gap pad is disposed between the aluminum encasement and the processing circuitry. A pressure sensitive adhesive is disposed between an internal surface of the titanium housing and the aluminum foil encasement, the pressure sensitive adhesive being in direct contact with the housing and with the aluminum encasement.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an inside view of a patient with a fully implantable blood pump constructed in accordance with the principles of the present application;

FIG. 2 is an inside perspective view of a controller of the fully implantable blood pump shown in FIG. 1;

FIG. 3 is an exploded view of a portion of the controller shown in FIG. 2;

FIG. 4 is a cross-sectional view of a first portion of the controller shown in FIG. 2;

FIG. 5 is a zoomed in view an inside corner of the controller shown in FIG. 2; and

FIG. 6 is a zoomed in view a portion of a thermally conductive encasement within the controller shown in FIG. 2.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

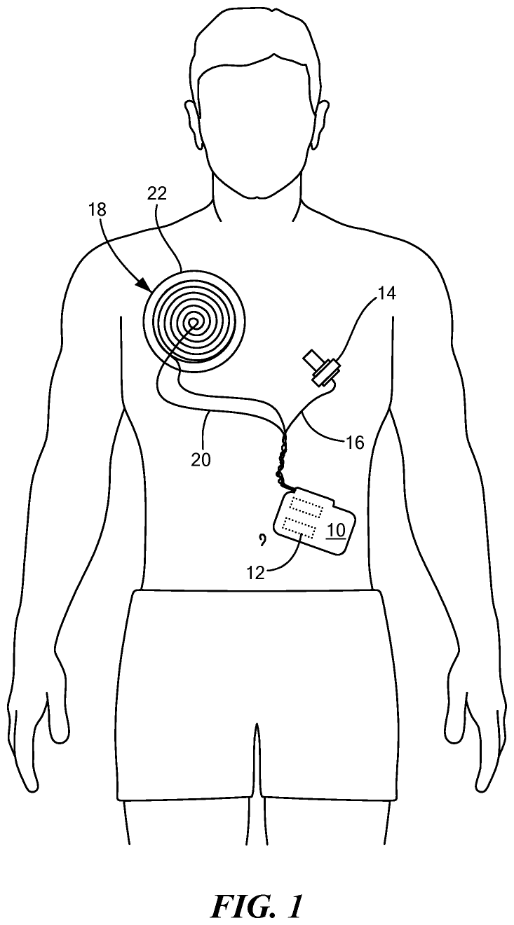

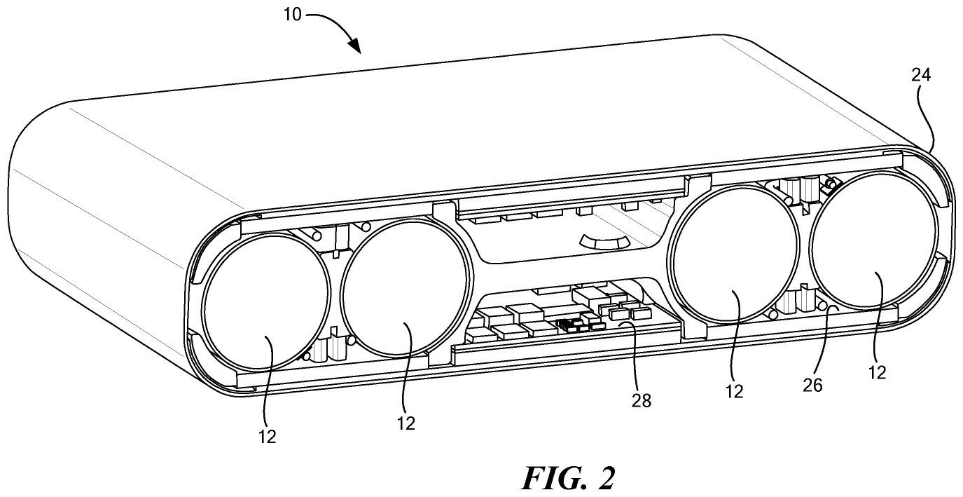

Referring to the drawings in which like reference designators refer to like elements, there is shown in FIGS. 1 and 2 an exemplary implantable controller for an implantable medical device constructed in accordance with the principles of the present application and designated generally as “10.” The controller 10 may include one or more batteries 12 configured to power the components of the controller and provide power to one or more implantable medical device, for example, a blood pump such as ventricular assist device (VAD) 14 implanted within the left and/or right ventricle of the patient's heart. VADs 14 may include centrifugal pumps, axial pumps, or other kinds electromagnetic pumps configured to pump blood from the heart to blood vessels to circulate around the body. One such centrifugal pump is the HVAD sold by HeartWare, Inc. and is shown and described in U.S. Pat. No. 7,997,854 the entirety of which is incorporated by reference. One such axial pump is the MVAD sold by HeartWare, Inc. and is shown and described in U.S. Pat. No 8,419,609 the entirety of which is incorporated herein by reference. In an exemplary configuration, the VAD 14 is electrically coupled to the controller 10 by one or more implanted conductors 16 configured to provide power to the VAD 14, relay one or more measured feedback signals from the VAD 14, and/or provide operating instructions to the VAD 14.

Continuing to refer to FIG. 1, a receiving coil 18 may also be coupled to the controller 10 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively coupled through the patient's skin by a transmission coil (not shown) powered by an external battery (not shown) disposed opposite the receiving coil 18 on the outside of the patient's body. The receiving coil 18 may be disposed within a hermetically sealed package 22 that does not interfere with the electromagnetic coupling between the receiving coil 18 and the external transmission coil (not shown) of the receiving coil 18.

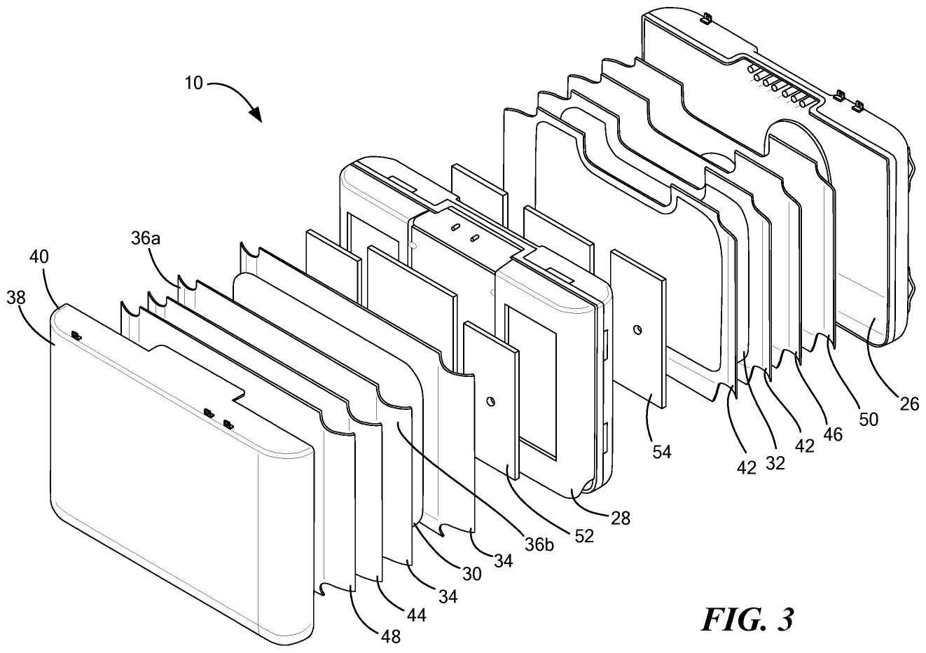

Referring now to FIGS. 2 and 3, the controller 10 defines a housing 24 sized and configured to enclose and retain the various components of the controller 10 discussed herein. The housing 24 is composed of a biocompatible metal or metal alloy, for example, titanium and in one configuration defines an elongated shape with a slim profile. In one configuration, the housing 24 hermitically seals the components within an enclosure 26 defining therein and in other configurations the housing does not hermetically seal the components therein. In the configuration shown in FIG. 2, the housing 24 includes and encloses four batteries 12, which may be rechargeable and configured to power the implanted medical device, for example, VAD 14. In the configuration shown in FIG. 2, the batteries 12 are separated into pairs with one or more circuit boards 28 disposed therebetween that receive power from the batteries 12. The circuit boards 28 may include processing circuitry having one or more processors configured to, among other things, operate the implantable medical device 14.

Continuing to refer to FIGS. 2 and 3, the controller 10 may further include a first piece of graphite 30 disposed within the housing 24. The first piece of graphite 30 may define a thin sheet that spans partially or substantially the entirety of the area of the controller 10. The first piece of graphite 30 may be entirely flat or may include a slight curvature at its ends. In one configuration, the first piece of graphite 30 graphite is annealed pyrolytic graphite (APG) and is configured to spread heat released by the internal electronics of the controller 10, for example, the circuit boards 28 and/or the one or more batteries 12. In one configuration, a second piece of graphite 32 is included within the housing 24, which includes the same or similar properties as the first piece of graphite 30. The second piece of graphite 32 may be disposed opposite the first piece of graphite 32 with the one or more batteries 12 and the circuit boards 28 disposed therebetween. In one configuration, the first piece of graphite 30 and/or the second piece of graphite 32 may be composed of any form of graphite, whether APG or amine functionalized graphene (AFG). In other configurations, the graphite may be substituted with other thermal spreaders such hexagonal boron nitride (hBN) within thermoplastic polyurethane (TPU)

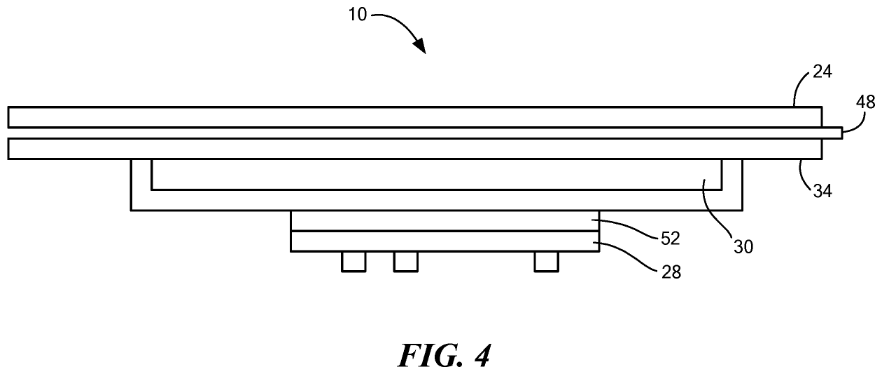

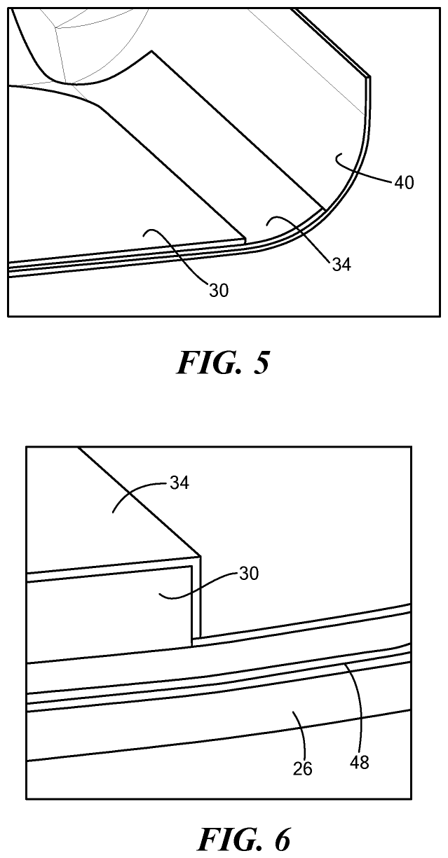

Referring now to FIGS. 2-5, surrounding or entirely enclosing the first piece of graphite 30 may be a first thermally conductive encasement 34. In one configuration the thermally conductive encasement is an aluminum encasement 34. In other configurations, the thermally conductive encasement may be polyimide or copper. The first encasement 34 is configured spread heat away from the controller 10. In one configuration, the first encasement 34 is aluminum foil laminated directly onto the first piece of graphite 30, although the first aluminum encasement 34 may define or otherwise include any form of aluminum or aluminum alloy. In other configurations, the encasement 34 may be composed of other materials, such as copper, steel, plastic, polyimide, or parylene. In one configuration, the first encasement 34 defines flared edges 36a and 36b on opposite ends. The flared edges 36a and 36b extend away from the first encasement 34 within the housing 24. The flared edges 36a and 36b may contour the curvature of an inner surface 38 of the housing 24. For example, as shown in FIGS. 4 and 5, the housing 24 may define arcuate corners 40 on both the exterior and the inner surface 38 of the housing 24. The flared edges 36a and 36b may be conformal with the inner surface 38 to further increase thermal spreading away from the interior of the controller 10. A second encasement 42 may be included within the housing 24 sized and configured to retain the second piece of graphite 32. In one configuration the encasement 42 is aluminum, and in other configurations, the encasement 42 may be composed of other materials, such as copper, steel, plastic, polyimide, or parylene. In one configuration, the battery 12 and the circuit board 28 having the processing circuitry are disposed between the first encasement 34 and the second encasement 42. The second encasement 42 may be sized and configured in the same or similar manner as that of the first encasement 34, but disposed the opposite side within the enclosure of the housing 24.

Continuing to refer to FIGS, 3-5, optionally, a first polyimide liner 44 may be disposed within the housing 24. The first polyimide liner 44 may define a sheet or other substantially planar material and is disposed between the first encasement 34 and the inner surface 38 of the housing 24. A second polyimide liner 46, similar or the same as the first polyimide liner 44 may further be disposed between the second encasement 42 and the inner surface 38 of the housing 24. The polyimide liners 44 and 46 are configured to further spread heat away from the interior of the housing 24, and in particular, away from their respective encasements 34 and 42. As with the encasements 34 and 42, the polyimide liners 44 and 46 may define flared edges commensurate in size and shape with the flared edges 36a and 36b of the encasements 34 and 42. To adhere the polyimide liners 44 and 46 to the inner surface 38 of the housing 24, a pressure sensitive adhesive (PSA) 48 may be included and disposed between the inner surface 38 and the polyimide liner 44 to adhere the polyimide liner 44 to the inner surface. As we with the polyimide liner 44, the PSA 48 may define flared edges commensurate in size and shape with the flared edges 36a and 36b of the encasements 34 and 42. A second PSA 50 may be included on the opposite of the housing 24 configured to adhere the second polyimide liner 46 to the inner surface 38. In one configuration, the first piece of graphite 30, the first polyimide liner 44, and the PSA each defines planes that are substantially parallel to each other.

Continuing to refer to FIGS. 3-5, a first thermally conductive gap pad 52 and a second thermally conductive gap pad 54 may be included within the housing 24 disposed between the respective encasements 34 and 42 and the circuit board 28 and the battery 12. In one configuration, the conductive gap pad is a soft, flat, conformable material, for example, graphite fibers embedded in a silicone resin to combine the high thermal conductive properties of graphite with the soft conformability of silicone polymer. The conductive gap pads 52 and 54 are configured to spread heat away from the circuit board 28 toward the respective encasements 34 and 42 and provide a high impedance electrical barrier to prevent circuitry from coming into electrical contact with the encasements 34 and 42. In one configuration, the conductive gap pads 52 and 54 are commensurate in area with the circuit board 28, but may be any shape any size.

Although the components of the controller 10 are described with respect to the controller 10, it is contemplated that the described components could be integrated into a medical device, for example a pacemaker, or any other medical device, whether implantable or non-implantable.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable medical device, comprising:
a metallic housing defining an enclosure, processing circuitry disposed within the enclosure and configured to control operation of the implantable medical device;
a first thermally conductive encasement disposed within the enclosure;
a first piece of graphite disposed within the first thermally conductive encasement; and
an adhesive disposed between an internal surface of the metallic housing and the first thermally conductive encasement.

2. The device of claim 1, wherein the first piece of graphite is of annealed pyrolytic graphite.

3. The device of claim 1, further including a battery disposed within the housing, wherein the battery is configured to provide power to the processing circuitry and to provide power to the implantable medical device.

4. The device of claim 3, wherein the enclosure further includes a second thermally conductive encasement and a second piece of graphite enclosed within the second thermally conductive encasement, wherein the battery and the processing circuitry are disposed between the first thermally conductive encasement and the second thermally conductive encasement.

5. The device of claim 4, further including a first conductive gap pad disposed between the processing circuitry and the first thermally conductive encasement.

6. The device of claim 1, wherein the metallic housing is composed of a biocompatible metal.

7. The device of claim 1, wherein the first thermally conductive encasement includes aluminum foil, and wherein the first piece graphite is laminated in the aluminum foil.

8. The device of claim 1, wherein the medical device is a controller for an implantable blood pump.

9. The device of claim 1, wherein the first piece of graphite defines a first plane and the adhesive defines a second plane substantially parallel to the first plane.

10. The device of claim 1, wherein the housing is sized to be implanted within a portion of a patient's torso.

11. An implantable controller for an implantable medical device, comprising:
a metallic housing defining an enclosure, the housing being sized to be implanted within a portion of a patient's torso
the enclosure containing processing circuitry configured to control the operation of the implantable medical device;
a first piece of graphite disposed within the housing, the first piece of graphite being enclosed within a first aluminum encasement, the first aluminum encasement defining a plurality flared aluminum edges extending from the encasement; and
a pressure sensitive adhesive disposed between an internal surface of the metallic housing and the first aluminum encasement, the pressure sensitive adhesive being in direct contact with the housing and with the first aluminum encasement.

12. The controller of claim 11, wherein the first piece of graphite is annealed pyrolytic graphite.

13. The controller of claim 11, further including a battery disposed within the housing, wherein the battery is configured to provide power to the processor and to provide power to the medical device.

14. The controller of claim 13, wherein the enclosure further includes a second aluminum encasement and a second piece of graphite enclosed within the second aluminum encasement, wherein the battery and the processing circuitry are disposed between the first aluminum encasement and the second aluminum encasement.

15. The controller of claim 14, further including a first conductive gap pad disposed between the processing circuitry and the first aluminum encasement.

16. The controller of claim 11, wherein the metallic housing is composed of a biocompatible metal.

17. The controller of claim 11, wherein the first aluminum encasement includes aluminum foil, and wherein the first piece of graphite is laminated in the aluminum foil.

18. The controller of claim 11, wherein the medical device is an implantable blood pump.

19. The controller of claim 11, wherein the first piece of graphite defines a first plane and the pressure sensitive adhesive defines a second plane substantially parallel to the first plane.

20. An implantable controller for an implantable blood pump, comprising:
a titanium housing defining an enclosure, the housing being sized to be implanted within a portion of a patient's torso;
the housing containing processing circuitry configured to control the operation of the implantable blood pump and a battery configured to provide power to the implantable blood pump;
a piece of graphite disposed within the housing, the piece of graphite being enclosed and laminated within an aluminum foil encasement, the aluminum foil encasement defining a plurality flared aluminum edges extending from the encasement and contouring a corresponding curved interior surface of the housing; and
a conductive gap pad disposed between the aluminum encasement and the processing circuitry; and
a pressure sensitive adhesive disposed between an internal surface of the titanium housing and the aluminum foil encasement, the pressure sensitive adhesive being in direct contact with the housing and with the aluminum encasement.
